(19) United States
(12) Patent Application Publication
(10) Pub. No.: US 2004/0079639 A1
(43) Pub. Date: Apr. 29, 2004

(54) System and method for biological fraction collection for capillary electrophoresis

(75) Inventors: Sakuichiro Adachi, Kokubunji (JP); Takashi Anazawa, Kodaira (JP); Takashi Irie, Musashimurayama (JP)

Correspondence Address: Stanley P. Fisher, Reed Smith LLP, Suite 1400, 3110 Fairview Park Drive, Falls Church, VA, 22042-4503, US

(73) Assignee: Hitachi, Ltd.

(21) Appl. No.: 10/443,991

(22) Filed: May 23, 2003

(30) Foreign Application Priority Data
Oct. 23, 2002 (JP) 2002-307793

(51) Int. Cl. C12M 1/34; G01F 1/64

(52) U.S. Cl. 204/451; 435/287.2

(57) ABSTRACT

A system and method are provided for fraction collection for capillary electrophoresis. In one example, the system includes a capillary configured to electrophoretically separate biological fractions into separated biological fractions, and further configured to discharge the separated biological fractions from a capillary end; a flow cell surrounding the capillary end, wherein the flow cell is configured to collect the separated biological fractions discharged from the capillary end into a collecting vessel; an irradiator configured to irradiate a light beam onto the capillary at a detection point, wherein the light beam causes emitted light from the capillary; and a detector configured to detect the emitted light.

### COPYRIGHT NOTICE

A portion of the disclosure of this patent document contains material which is subject to copyright protection. The copyright owner has no objection to the facsimile reproduction by anyone of the patent document or the patent disclosure, as it appears in the Patent and Trademark Office patent file or records, but otherwise reserves all copyright rights whatsoever.

### BACKGROUND OF THE INVENTION

1. Field of the Invention

The present invention generally relates to analysis of biological fractions, such as deoxyribonucleic acid (DNA) strands, ribonucleic acid (RNA) strands, and any type of protein. More particularly, the present invention relates to biological fraction collection for capillary electrophoresis.

2. Discussion of Background

Since the advent of gene sequencing technology, techniques for analyzing biological materials have become increasingly more important. Techniques for analyzing expression genes have attracted special attention because these techniques are considered to be useful in the technologies related to gene diagnosis, gene therapy, and new drug development.

Techniques for analyzing expression genes involve separating a DNA sample using slab gel electrophoresis, collecting target DNA fractions by gel cutting, and analyzing gene sequences. However, this type of collection cycle involving separation and collection steps is time consuming and requires a lot of skill. It also has a problem of low accuracy and separation performance with gel cutting. To overcome this problem, it is desired that collecting the biological fractions are automatically separated at a higher speed and accuracy by capillary-electrophoresis. The high separation performance of capillary-electrophoresis allows the biological fractions to be collected at a level of single nucleotide, resulting in various applications including direct sequencing.

In a case in which the biological fractions are collected by capillary electrophoresis, the vessel for collecting the biological fractions is disposed on the side of the end of the capillary, from which a sample is injected. After separation by capillary electrophoresis, the separated fractions are eluted out from the capillary end reach the collecting vessel, where the separated biological fractions are collected.

FIG. 1 shows a typical aspect of configuration of a main part of a conventional system for biological fraction collection for capillary electrophoresis. A capillary end 2 of a capillary 1 is inserted into a flow cell 3, which is then filled with a sheath liquid 4. The sheath liquid 4 is supplied into the flow cell 3 from a sheath liquid transfer tube 9 and discharged from the flow cell 3 through a collecting tube 6. Accordingly, a sheath flow is formed within the sheath liquid transfer tube 9, the flow cell 3, and the collecting tube 6. The flow velocity of the sheath flow 16 is kept constant at a high accuracy with almost no change with time. The sample is separated into sample fractions 5 by electrophoresis in the capillary 1 filled with a polymer solution 104 and detected at a detection point 401. Then, the sample fractions eluted from the capillary end 2 pass through the flow cell 3 and the collecting tube 6 together with the sheath flow 16 and are discharged from the end of the collecting tube, a collection point 8.

In general, a waste vessel 14 is disposed at the collecting point 8 to collect the sheath liquid 4 discharged from the collecting tube 6, wherein the target sample fractions 5 are collected by the waste vessel 14 disposed at the collecting point 8 is changed to a collecting vessel 7 in exact time with the time the fractions 5 reach the collection point 8. The timing of the change between the vessels depends on the position of the detection point 401. Two types of conventional collection methods are described below.

(a) A method comprising an on-column detection process, in which a laser beam 302 is irradiated directly onto the capillary 1 outside of the flow cell 3, the sample fractions 5 are eluted from the capillary end 2, and then the sample fractions 5 are moved to the collection point 8 together with the sheath flow 16 for collecting the sample fractions 5. The system configuration is the same as that shown in FIG. 1. For additional detail, see Karger et al., Anal. Chem. 1995, 67, 2974-1980.

(b) A method comprising a sheath flow detection process, in which after the sample fractions 5 are eluted from the capillary end 2, the laser beam 302 is irradiated onto the sample fractions 5 in the sheath liquid 4 in the flow cell 3 for fluorescence-detection and the sample fractions 5 are moved together with the sheath flow 16 to the collection point 8 for collection. FIG. 2 shows the system configuration. For additional detail, see Irie et al., Electrophoresis 2000, 21, 367-374.

To collect the sample fractions at high separation performance, it is required that the timing of the change from the waste liquid vessel 14 disposed at the collection point 8 to the collecting vessel 7 is estimated at a high accuracy. For this purpose, the traveling time required for the sample fractions 5 to reach from the detection point 401 to the collection point 8 should be measured at a high accuracy.

The method (a) calculates the traveling time required for the sample fractions 5 to reach the collection point 8 by finding the sum of the elution time required for the sample fractions 5 to be eluted, during which the sample fractions 5 are forced into electrophoresis from the detection point 401 to the capillary end 2, and the time required for the sample fractions 5 to flow together with the sheath flow 4 from the capillary end 2 to the collection point 8. The time required to be eluted can be obtained by dividing the distance between the detection point 401 to the capillary end 2 by an electrophoretic velocity obtained from measurement. The electrophoretic velocity can be obtained as an average electrophoretic velocity along the length of the capillary 1 by dividing the electrophoretic distance (between the detection point to the end of the capillary) by the detection time required for the sample fractions 5 to be forced into electrophoresis from the sample-injection end of the capillary to the detection point 401. However, the electrophoretic velocity changes depending on the state of a polymer solution 104 and empirically, a deviation of ±2% from the measured value has been known. For this reason, an elution-time error increases proportional to the distance between the detection point 401 and the capillary end 2. Unfortunately, the reduction of the distance is technically difficult. Consequently, the traveling time can not be determined at a high accuracy.

The method (b) measures the traveling time by dividing the distance the detection point 401 to the collection point 8 by a flow rate of the sheath flow 16. Since the flow rate of the sheath flow 16 can be kept constant at a high accuracy with almost no change with time, the time required for the sample fractions 5 to flow together with the sheath flow can be determined at a high accuracy.

Unfortunatley, the method (b) has a problem of deteriorated separation performance at the collection point 8, which is caused due to diffusion of the sample fractions 5 into the sheath liquid 4 during the time required for the sample fractions 5 to flow together with the sheath flow. Although deterioration in separation performance due to diffusion can be minimized by increasing the flow rate of the sheath flow 16 to decrease the time required for the sample fractions 5 to flow together with the sheath flow, sample fractions can not be collected at an accuracy of single nucleotide because the flow rate of the sheath flow can not be increased above a certain point from an aspect of sensitivity.

### SUMMARY OF THE INVENTION

It has been recognized that what is needed is a method for collecting DNA fractions of single nucleotide at a high accuracy. Broadly speaking, the present invention fills these needs by providing a system and method for biological fraction collection for capillary electrophoresis. It should be appreciated that the present invention can be implemented in numerous ways, including as a process, an apparatus, a system, a device or a method. Several inventive embodiments of the present invention are described below.

In one embodiment, a system is provided for fraction collection for capillary electrophoresis. The system comprises a capillary configured to electrophoretically separate biological fractions into separated biological fractions, and further configured to discharge the separated biological fractions from a capillary end; a flow cell surrounding the capillary end, wherein the flow cell is configured to collect the separated biological fractions discharged from the capillary end into a collecting vessel; an irradiator configured to irradiate a light beam onto the capillary at a detection point, wherein the light beam causes emitted light from the capillary; and a detector configured to detect the emitted light.

In another embodiment, a method is provided for fraction collection for capillary electrophoresis. The method comprises separating biological fractions in a capillary into separated biological fractions; discharging the separated biological fractions from the flow cell by injecting a sheath fluid into the flow cell surrounding the capillary end; irradiating a light beam at a detection point, wherein the light beam cause emitted light from the capillary; detecting the emitted light; and collecting the separated biological fractions discharged from the flow cell into individual collecting vessels.

The invention encompasses other embodiments of a method, a system, and an apparatus, which are configured as set forth above and with other features and alternatives.

### BRIEF DESCRIPTION OF THE DRAWINGS

The present invention will be readily understood by the following detailed description in conjunction with the accompanying drawings. To facilitate this description, like reference numerals designate like structural elements.

FIG. 1 is a view showing the configuration of the main part of the fraction collection system for capillary electrophoresis using the conventional on-column detection;

FIG. 2 is a view showing the configuration of the main part of the fraction collection system for capillary electrophoresis using the conventional sheath flow detection.

FIG. 3 is a view showing the configuration of the main part of the fraction collection system for capillary electrophoresis according to the present invention;

FIG. 4 is a view showing the result of fluorescent measurement of DNA fractions separated based on their lengths through capillary electrophoresis, in accordance with an embodiment of the present invention;

FIG. 5 is a view showing the configuration of the main part of the fraction collection system for capillary electrophoresis using two-point detection, in accordance with an embodiment of the present invention;

FIG. 6 is a view showing the scale of the elution time prediction error and the relationship between the detection point and the capillary end, in accordance with an embodiment of the present invention;

FIG. 7 shows the configuration of the system for biological fraction collection, in accordance with a first embodiment of the present invention;

FIG. 8 shows the details of the sample-injected side unit of the system for fraction collection, in accordance a first embodiment of the present invention;

FIG. 9 shows a detailed view of the end-side unit of the system for fraction collection, in accordance with a first embodiment of the present invention;

FIG. 10 is a schematic elevational view of the detection-part unit of the system for fraction collection, in accordance with a first embodiment of the present invention;

FIG. 11 is a schematic view of the detection unit shown in FIG. 10 as seen from the right side, in accordance with a first embodiment of the present invention;

FIG. 12 is a view illustrating the procedure for collecting biological fractions, in accordance with a first embodiment of the present invention;

FIG. 13 shows the configuration of the detection-part unit 300 according to the second embodiment of the present invention;

FIG. 14 is a view illustrating the procedure for collecting biological fractions according to the second embodiment of the present invention;

FIG. 15 is a view showing the details of the detection-part nit according to a third embodiment of the present invention;

FIG. 16 is a view showing the configuration of the main part according to a third embodiment of the present invention;

FIG. 17 is a view showing the intensity of the background spectrum when an angle defined between the wavelength-dispersion directions and the proceedings of the laser beam is changed according to the third embodiment of the present invention;

FIG. 18 is a view showing the configuration of the apparatus according to a fourth embodiment of the present invention;

FIG. 19 is a view showing the details of the sample-injected side unit according to a fourth embodiment of the present invention;

FIG. 20 is a view showing the configuration of the main part of the system for fraction collection according to a fourth embodiment of the present invention;

FIG. 21 is a view showing the details of the capillary end side unit according to a fourth embodiment of the present invention;

FIG. 22 is a view showing the configuration of the main part of the system for fraction collection according to a fifth embodiment of the present invention; and

FIG. 23 is a view showing the details of the detection-part unit according to a fifth embodiment of the present invention.

### DESCRIPTION OF THE PREFERRED EMBODIMENTS

An invention for a system and method for fraction collection for capillary electrophoresis is disclosed. Numerous specific details are set forth in order to provide a thorough understanding of the present invention. It will be understood, however, to one skilled in the art, that the present invention may be practiced without some or without all of these specific details.

General Overview

FIG. 3 shows the inside of the flow cell 3 of a capillary electrophoresis apparatus, in accordance with an embodiment of the present invention. The present invention uses on-column detection, in which the laser beam 302 is irradiated directly onto the capillary 1 inside of the flow cell 3 for fluorescence detection, and after the sample fractions 5 are discharged from the capillary end 2, they are moved to the collection point 8 together with the sheath flow 16 for collection.

The method of the present invention allows the distance between the detection point 401 and the capillary end 2 to be reduced as possible, which enables the elution-time error to be decreased, resulting in high-accuracy prediction of the traveling time. At this point, the prediction accuracy for the traveling time required to collect DNA fractions at a high accuracy is estimated.

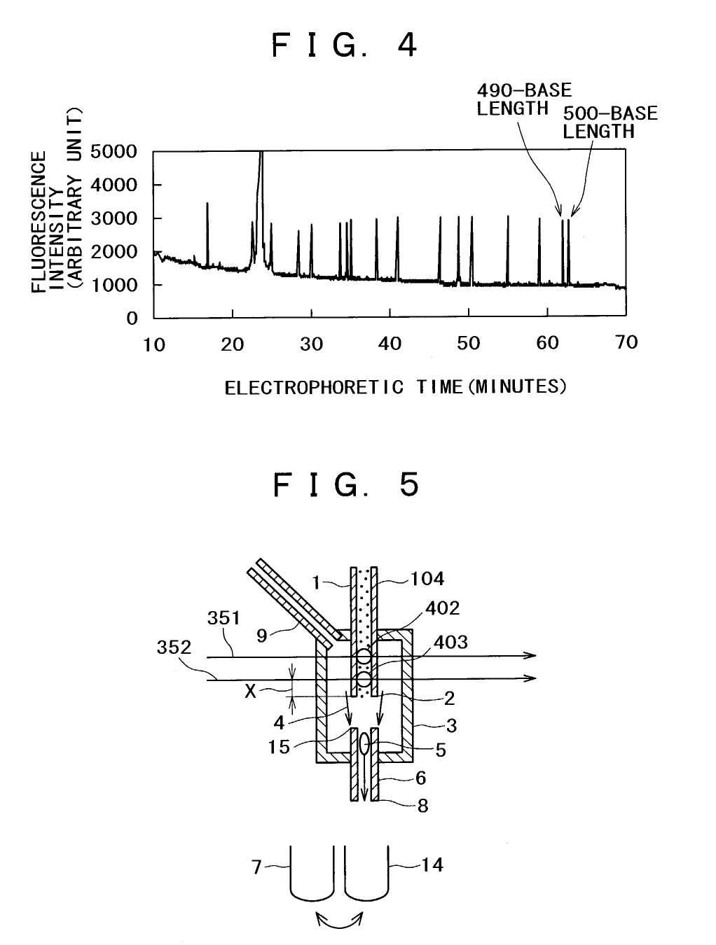

FIG. 4 shows results of a fluorescence measurement, for which electrophoresis was conducted using POP6 for a polymer solution and GeneScan 500 Rox from Applied Biosystems for a sample 101 under the conditions of the distance of 360 mm between the sample-injected end of the capillary 1 and the detection point, a temperature of 50° C., and a voltage of 15 kV. The time required to detect 490-base length DNA fractions was 61.9 minutes and the time required to detect 500-base length DNA fractions was 62.7 minutes. The difference between these measured times was about 48 seconds (0.8×60). From this value, a detection time interval/base 4.8(sec./base) can be obtained from the formula 48(sec.)/10(bases) for this base length DNA fractions. In the light of the calculation method, to forecast the traveling time at an accuracy of single nucleotide, the time can be preferably forecast at an accuracy within ±0.5 sec.

In the method of the present invention, the detection point 401 can be set at the closest point to the capillary end, resulting in the reduced traveling-time error.

To collect the biological fractions at a higher accuracy, the method, in which the secondary detection point is set, may be considered as shown in FIG. 5. In the method shown in FIG. 3, since an average electrophoretic velocity along the length of the capillary 1 is measured, the measured value is affected by a difference in temperature. On the contrary, in the case that detection is performed at two points in the vicinity of the capillary end 2 to measure an average of electrophoretic velocity values obtained the distance between the two points (the two-point detection method) as shown in FIG. 5, when the temperature in the vicinity of the capillary end 2 is adjusted so that it is kept uniform, the measured values are almost not affected by the difference in temperature depending on the position of the capillary, which enables the electrophoretic velocity to be measured at a higher accuracy. Note that even if the two-point detection method is used, a certain degree of deviation may develop in electrophoretic velocity. It is considered that this deviation may be induced by the presence of dust or minute air bubbles in the polymer solution 104 and empirically, the scale of deviation is assumed to be ±2% in terms of electrophoretic velocities. For this reason, the distance (the distance X in FIG. 5) between the detection point and the capillary end should be reduced for two-point detection as well.

A predicted elution-time error is considered for the two-point detection method. As known from FIG. 5, first and second detection points 402 and 403 are set at the capillary 1 inside of the flow cell 3. Assuming that the length between the second detection point 403 and the capillary end 2 is x, the average of electrophoretic velocity values obtained at the two points is v, and a deviation in electrophoretic velocity is ±2%, the scale of the error, which may develop in predicting the elution time required for the fractions to be forced into electrophoresis at an interval of x, can be expressed by:

|Δt|=(1/v−1/1.02v)x Equation 1

In Equation 1, when |Δt| is equal or less than 0.5 sec., the DNA fractions can be collected under separation performance at an accuracy of single nucleotide. To reduce |Δt| in the expression, it is preferable that the distance x is decreased.

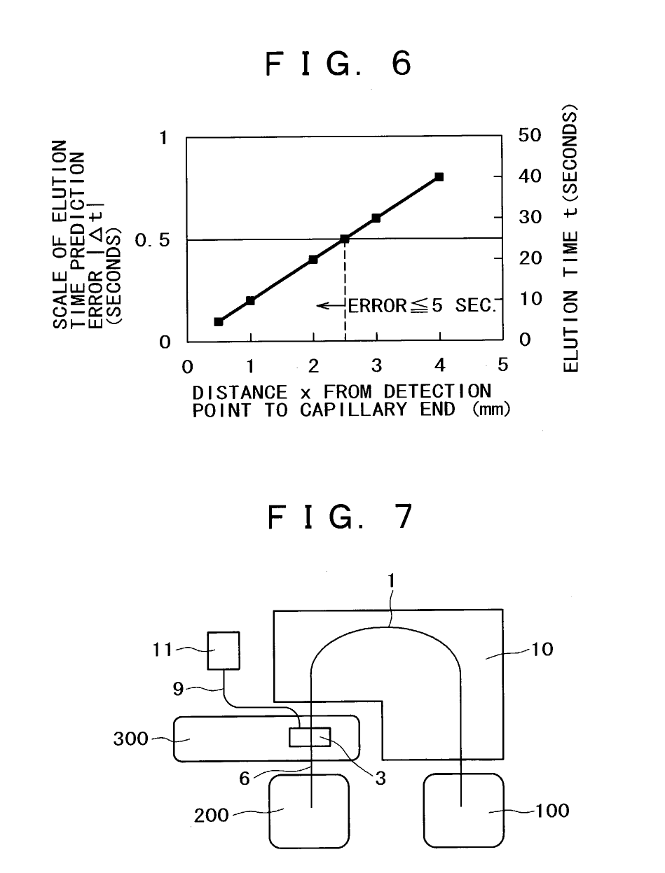

Now, it is assumed that 500-base length DNA fractions are collected. With reference to FIG. 4, with regards to the conditions for electrophoresis illustrated, the distance between the sample-injected end of the capillary 1 and the detection point is 360 mm and the detection time required for the 500-base length DNA fractions to be detected is 62.7 min. When these values are assigned into the formula (360/62.7/60), a value, about 0.10 mm/s can be obtained. FIG. 6 plots the scale |Δt| of the elution-time prediction error and the elution time t relative to the distance x. Since Δt increases proportional to x, the distance x must be equal to or less than 2.6 mm to keep |Δt| equal to or less than 0.5 sec.

If the apparatus as shown in FIG. 4 instead of that as shown in FIG. 5, which is referred to for illustration mentioned above, is referred to, namely the one-point detection method is focused on, x indicates the distance between the detection point 401 and the capillary end 2. In this case, the distance x must be equal to or less than 2.6 mm to keep |Δt| equal to or less than 0.5 sec.

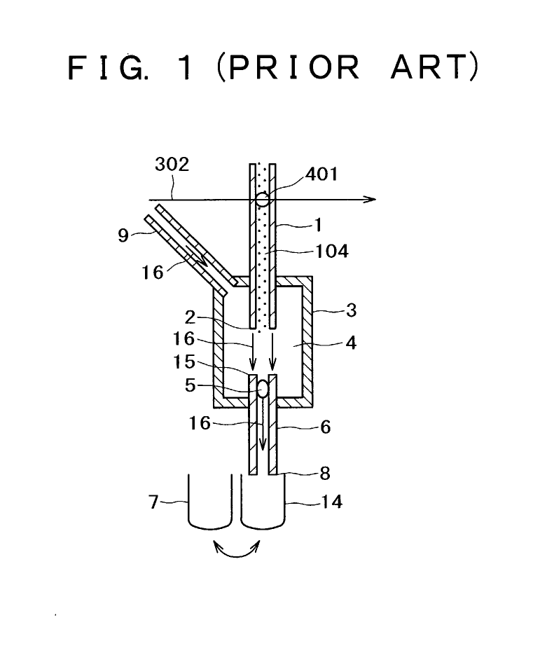

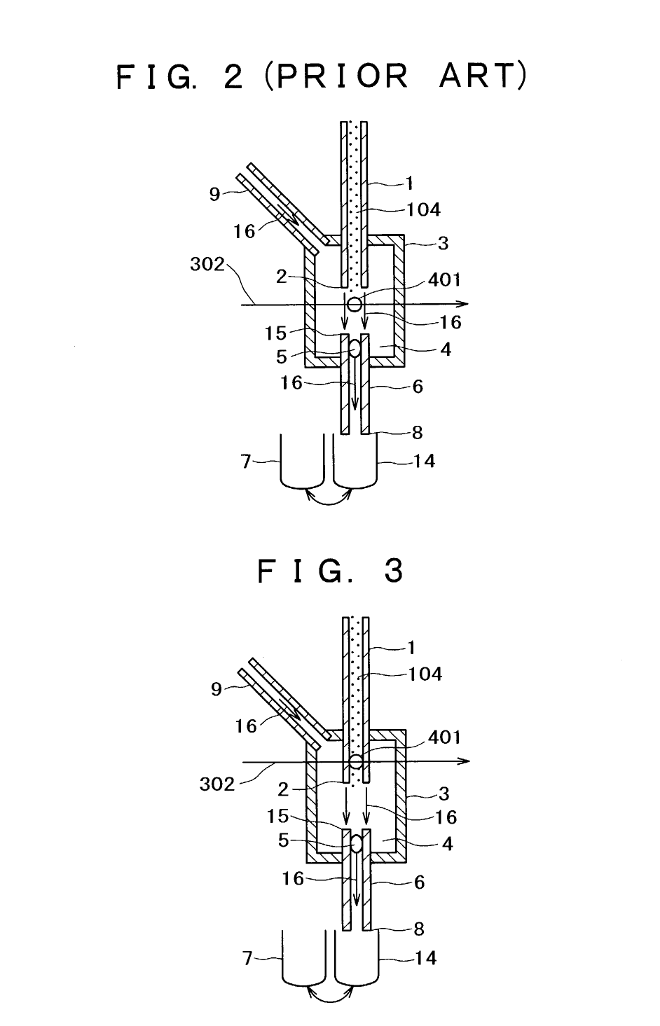

On the contrary, in the conventional method, which uses on-column detection outside of the flow cell 3 as shown in FIG. 1, it is difficult to keep x equal to or less than 2.6 mm from an aspect of arrangement of the detection system and vessels. In this case, x indicates the distance between the detection point 401 and the capillary end 2. In fact, in the conventional method (Karger et al, Anal. Chem. 1995, 67, 2974-2980), x is 10.0 (mm) and the elution-time prediction error is as much as about ±2.0 sec. taking the calculation mentioned above into account. Since the accuracy for predicting the elution time required for collection at the accuracy of single nucleotide is ±0.5 sec., the elution time cannot be predicted at an accuracy of single nucleotide in the conventional method.

By irradiating the laser beam directly onto the capillary 1 inside of the flow cell 3 according to an embodiment of the present invention for on-column detection, the electrophoretic distance between the detection point 401 and the capillary end 2 can be reduced. This allows the elution-time prediction error to be decreased, enabling the traveling time from the detection point 401 to the collection point 8 to be obtained at a high accuracy.

In addition, since the DNA fractions are detected on the capillary 1, the detection sensitivity is not affected by the flow rate of the sheath flow 16, even if it is increased. For this reason, the influence of diffusion can be minimized and the DNA fractions may be collected under high separation performance. Setting two or more detection points 401 allows the electrophoretic velocity to be predicted at a higher accuracy, improving the accuracy for the traveling time.

Note that although fluorescence detection is described above, it is, of course, the absorption may be detected by using a deuterium lamp or an ultraviolet light source such as an ultraviolet laser, irradiating an ultraviolet light beam onto the detection point 401 and measuring the amount of light passed.

Although the method mentioned provides an example of separation of the DNA fractions, the method may be applied to separation of the RNA fractions or any kind of proteins.

As mentioned above, the present invention provides the system, which can collect individual separated bases with an elution-time error of ±0.5 sec., by performing on-column detection inside the flow cell to ensure the distance x≦2.6 mm. This saves labor for refining the separated fractions in the succeeding process, for example, if DNA fractions with only one different base can be collected, direct sequencing can be performed using the collected fractions.

First Embodiment

A first embodiment has been designed so that the DNA sample mixed with a plural of DNA fractions of different base-lengths marked with a fluorescent dye is used as a biological material sample and the DNA fractions are separated based on their base lengths by capillary-electrophoresis for collection.

FIG. 7 shows the configuration of the system for biological fraction collection, in accordance with a first embodiment of the present invention. The apparatus comprises a capillary 1, a temperature adjustment plate 10, a sheath vessel 11, a sheath liquid transfer tube 9, a flow cell 3, a collecting tube 6, a sample-injected side unit 100, an end-side unit 200, and a detection-part unit 300. The sample is injected into the capillary 1 from the sample-injected side unit 100, is forced into electrophoresis toward the end-side unit 200 when a voltage is applied at the both ends of the capillary 1, and separated into sample fractions 5 based on their electrophoretic velocities. The sample fractions 5 are detected at the detection-part unit 300 and then collected at the capillary end-side unit 200.

A polyimide-coated quartz tube (Polymicro) with an outer diameter of 360 μm, an internal diameter of 50 μm, a length of 360 mm is used as the capillary 1. In this embodiment, the quartz tube with a round cross section is used as the capillary 1 but the quartz tube with a square cross section may be used, instead. The temperature is adjusted at the center of the capillary 1 using the temperature adjustment plate 10. The temperature is set to 50° C.

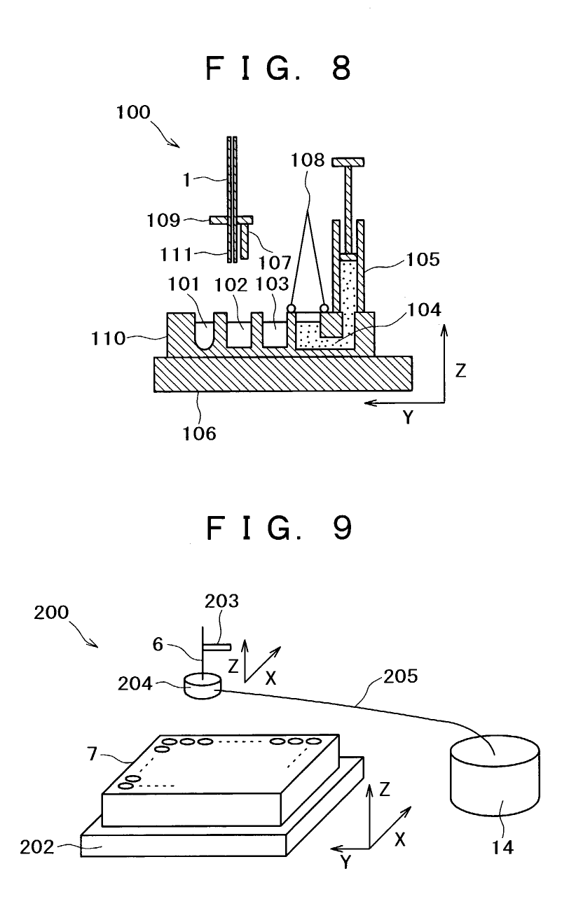

FIG. 8 shows the details of the sample-injected side unit 100, in accordance a first embodiment of the present invention. With reference to the FIG. 8, a sample 101, a buffer 102, pure water 103, a polymer solution 104, and a syringe 105 are prepared on a vessel 110 and the vessel 110 is disposed on a base plate 106, which is movable in the Y and Z directions. As the sample 101, a solution mixed with 8 kinds of DNA fragments of 161-, 201-, 271-, 363-, 364-, 421-, 489-, and 562-base lengths, which are marked with a fluorescent dye, is used. The concentration of each solution is 10 f mol/μl. A 3700 buffer is used from Applied Biosystems is used as the buffer 102. POP6 from Applied Biosystems is used as the polymer solution 104. A negative electrode is disposed in the vicinity of a sample-injected end 111 of the capillary 1.

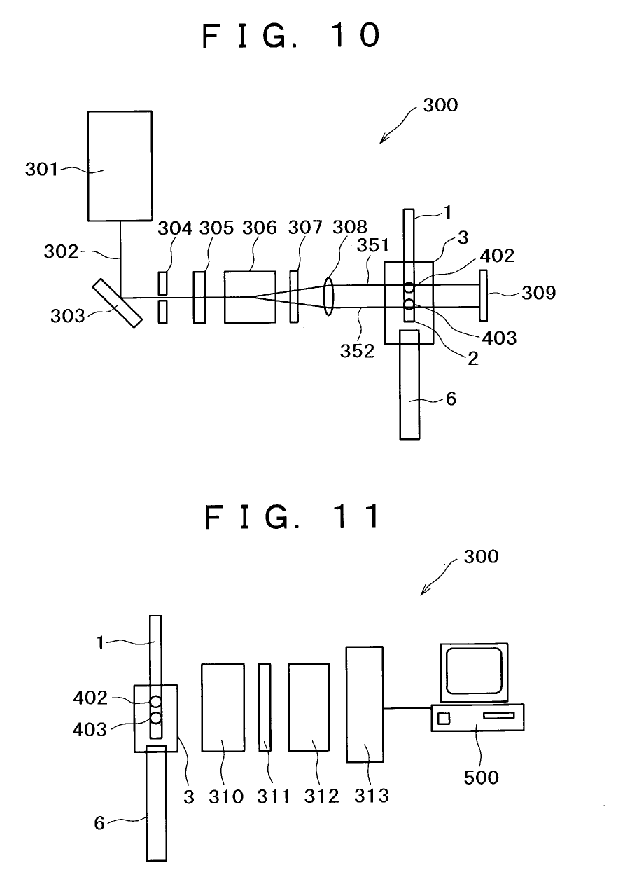

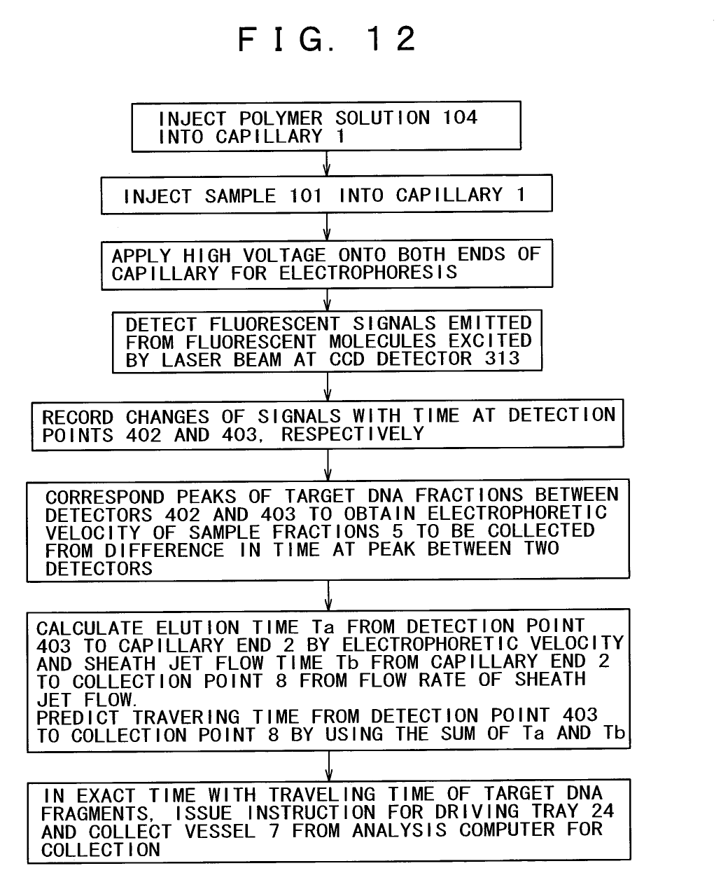

FIG. 9 shows the details of the end-side unit of the system for fraction collection, in accordance with a first embodiment of the present invention. The collecting tube 6 is connected to a ground electrode 203 through a stainless-steal hollow tube with a length of 70 mm and an internal diameter of 500 μm. The ground electrode to be used must be electrically connected to the ground constantly. A tray 204 and a collecting vessel 7 are disposed under the collecting tube 6. The tray 204 is movable in the X and Z directions and connected to a waste liquid vessel 14 through a waste liquid tube 205. The collecting vessel 7 is disposed on a collecting vessel base 202, which is movable in the X, Y, and Z directions. A 384-well microtiter plate (384 Plate from Applied Biosystems) is used as a collecting vessel 7. In this embodiment, eight kinds of sample fractions 5 are collected and the apparatus is driven so that individual kinds are collected in their own wells.

The configuration inside of the flow cell 3 is the same as that shown in FIG. 5. The flow cell 3 is made of quartz and the internal cross section is 1 mm×1 mm. The polyimide coating film is scraped off from an interval of 10 mm from the capillary end 2 of the capillary 1 for setting two detection points 402 and 403. The distance between the first detection point 402 and the second detection point 403 is 5 mm and the distance between the second detection point 403 and the capillary end 2 is 2.5 mm. The distance between the capillary end 2 and the collecting tube inlet 15 is 2 mm. To excite fluorescent molecules of the sample fractions 5 at each detection point, a first laser beam 351 and a second laser beam 352 are irradiated onto the first and second detection points 402 and 403 through the flow cell 3 and the capillary 1. PoP6 from Applied Biosystems is used as the sheath liquid 4. The flow rate of the sheath liquid is 1 μl/sec.

FIG. 10 is a schematic elevational view of the detection-part unit 300 of the system for fraction collection, in accordance with a first embodiment of the present invention. The proceeding direction of the laser beam 302 emitted by a laser source 301 is adjusted using a mirror 303 so that it is guided into a Wollaston prism 306 through a pinhole 304 and a half-wave plate 305. The laser beam is split into two laser beam components by the Wollaston prism 306 with a separation angle of 5°. The first and second laser beams 351 and 352, after split into two laser beam components, are condensed into the capillary 1 inside of the flow cell 3 through a shutter 307 using a condenser 308. The distance between the center of the Wollaston prism 306 and the condenser 308 is the same as a focal distance of the condenser 308. To secure safety, the first and second laser beams passing through the capillary 1 is guided into a laser stopper 309, preventing the beams from reflecting or scattering outside. Sapphire 488-20 from Coherent is used as the laser source 301. A laser beam wavelength is 488 nm and its laser intensity is 20 mW. A beam separator product with a separation angle of 5° from Sigma Optical Machine is used as Wollaston prism 306. A lens with a diameter of 15 mm and focal distance of 40 mm is used as the condenser 308.

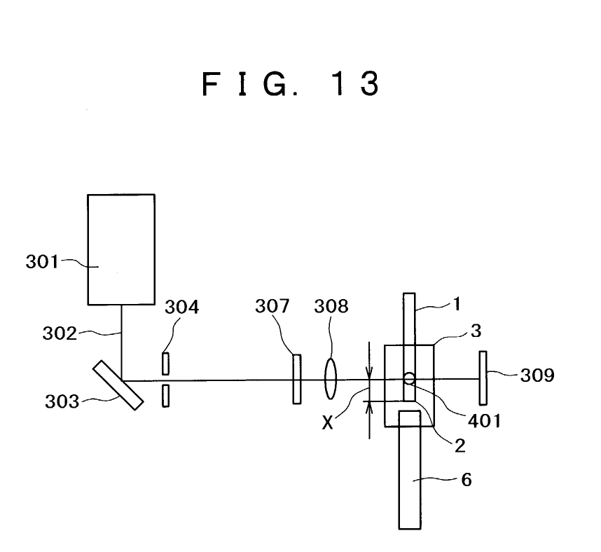

FIG. 11 is a schematic view of the detection unit 300 shown in FIG. 10 as seen from the right side, in accordance with a first embodiment of the present invention. One objective camera lens 310 is used for the first and second detection points 402 and 403 so that the detection unit 300 receives fluorescent light from the two detection points. Olympus F 1.2 with a focal length of 50 mm is used as the objective camera lens. The fluorescent beams from the two detection points, after condensed into a parallel flux through the objective camera lens 310, transmits a notch filter 311 and an image is formed on a charge-coupled device (CCD) detector 313 through an imaging camera lens 310. SuperNotch-Plus 488 nm from KAISER is used as the notch filter 311. This notch filter shields the laser beams from the first and second laser beams 351 and 352.

Alternatively, a long pass filter, which cuts a laser wavelength and transmits the fluorescent wavelength may be used instead of the notch filter 311. Nikon F1.4 with a focal length of 50 mm is used as an imaging camera lens 312 and a CCD from Andor as the CCD detector 313. The signals detected at the CCD detector 313 are transferred to an analytical computer 500 for analysis.

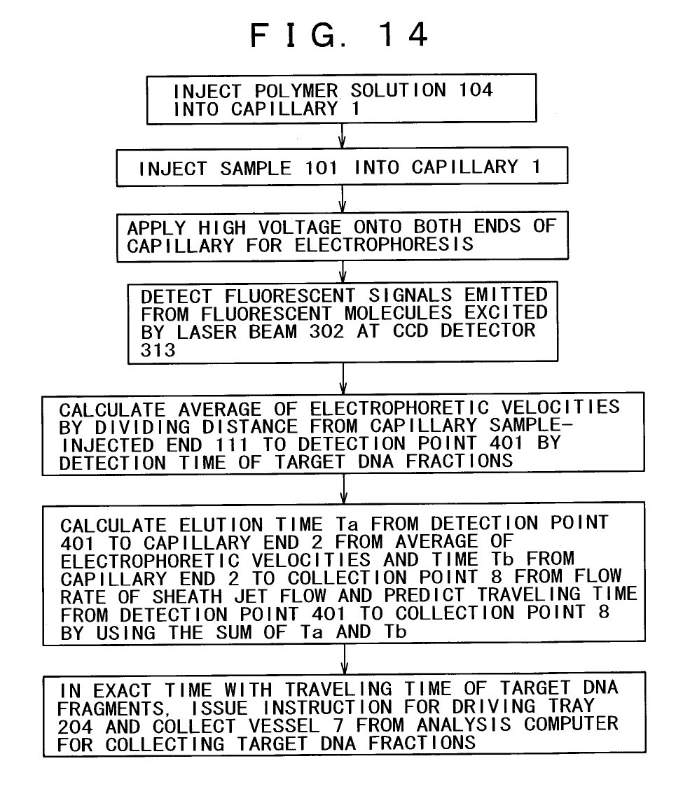

FIG. 12 is a view illustrating the procedure for collecting biological fractions, in accordance with a first embodiment of the present invention. At a sample-injected side unit 100, a sample-injected end 111 of the capillary 1 is submerged into the polymer solution 104 in the sample vessel 110 and a polymer solution 104 is pressurized using a syringe 105 to fill the capillary 1 with it. During the filling process, a O ring 108 makes a polymer filling plug 109 hermetically seal, ensuring to fill the pressurized polymer solution 104 in the capillary 1. An amount of polymer solution/filling is 5 μl. Any excess polymer solution 104, if filled, is discharged from the capillary end 2 of the flow cell 3 and removed away together with the sheath flow 16 through a collecting tube 6. When the polymer solution 104 is filled from the capillary end 2, the polymer solution is supplied through the flow cell 3. In this case, it is difficult to fill the pressurized polymer solution 104 because the flow cell 3 has not only the capillary 1 but also the collecting tube 6 and the sheath liquid transfer tube 9 connected. As shown in the embodiment, polymer filling from the sample-injected side allows the polymer solution 104 to be easily pressurized and filled.

After polymer filling, the sample-injected end 111 of the capillary 1 and a negative electrode 107 are submerged in pure water 103 for cleaning and then soaked into the sample 101. A ground electrode of an end-side unit 200 is connected to the ground and a voltage is applied onto both end of the capillary 1 by applying −1 kV voltage on the negative electrode 107. Since DNAs are negatively charged, the DNA sample migrates from the sample-injected side toward the end-side and injected into the capillary 1. The sample 101 is injected into the capillary 1 by applying voltage for 15 sec. After sample injection, the sample-injected end 111 and the negative electrode 107 are submerged in the pure water 103 and then in the buffer 102. The ground electrode 203 of the end-side unit 200 is connected to the ground and a voltage is applied to both ends of the capillary 1 by applying −15 kV voltage to the negative electrode 107 for electrophoresis. The DNA sample is separated into the sample fractions 5 depending on their base lengths through electrophoresis.

The first and second laser beams 351 and 352 are irradiated onto the first and second detection points 402 and 403, respectively, and excite on the fluorescent molecules marked with the DNA fractions while the sample fractions 5 pass through the detection points. The fluorescent luminescent light from the sample fractions 5 detected at the CCD detector 313 and the detected signals are transferred to an analytical computer 500. Changes of the signals at each detection point are recorded. When the target DNA fractions are forced into electrophoresis and detected at the first and second detection points at times t₁ and t₂, respectively, an average V₁ of electrophoretic velocities can be obtained by Δd/(t₂−t₁), which means that V₁ is equal to the distance Δd between the detection points divided by a difference in time (T₂−T₁) between two detection points. Assuming that the distance x between the second detection point 403 and the capillary end 2 is x, the elution time Tₐ can be obtained by x/V₁. The tₐ values can be obtained for each kind of DNA fragments with different base lengths.

For example, T₂ is 3100 sec. and T₁ is 3050 sec. In this case, assignment of Δd=5 mm in x/(T₂−T₁) yields a electrophoretic velocity V₁ of 0.1 mm/sec. (5/(3100−3050)). Since x is a.5 mm, the calculated Tₐ value is 25 sec. (2.5/0.1).

The DNA fractions, after eluted from the capillary end 2, pass through inside the flow cell 3 together with the sheath flow 16, and reach the collection point 8 through the collecting tube 6. It is assumed that the time required for the DNA fractions to move from the capillary end 2 to the collection point 8 is the sheath flow time T_{b}. T_{b} can be divided into T_{b1}, the time during which the DNA fractions pass through the flow cell 3, and T_{b2}, the time during which they pass through the collecting tube 6. Since the inner cross section of the flow cell 3 is 1 mm×1 mm and the flow rate of the sheath flow 16 is 1μ/sec., an average of flow rates of the sheath flow 16 is 1 mm/sec. Since the distance between the capillary end 2 and the collecting tube inlet 15 is 2 mm, T_{b1} of 2 sec. can be obtained from 2(mm)/1(mm/sec.).

On the other hand, since the inner diameter of the collecting tube is 500 μm, an average of flow rates of the sheath flow 16 passing through the collecting tube 6 is 5.1 mm/sec. (1/(0.25×0.25×3.14)). Since the length of the collecting tube 6 is 70 mm, T_{b2} is 13.7 sec (70/5.1).

Accordingly, T_{b} is 15.7 sec. (T_{b1}+T_{b2}=2+13.7).

Thus, the time required to traveling from the second detection point 403 to the collecting point 8, the traveling time, is expressed by Tₐ+T_{b}, yielding 38.7 sec. (25+13.7).

Note that these analyses are performed at an analysis part (the analytical computer 500).

The sheath liquid 4 discharged from the collecting tube 6 is generally guided into an external waste liquid vessel 14 from a tray 204 through a waste liquid tube 205. When the DNA fractions are collected, a driving instruction is issued by the analytical computer 500 and the tray 204 is changed to the collecting vessel 7 disposed at the collection point 8 in time with the traveling time of the target DNA fractions to collect the sheath liquid 4 together with the target DNA fractions in one of wells on the 384 plate of collecting vessel 7. In this embodiment, the time required to change from the tray 204 to the collecting vessel 7 and vice versa is 5 sec. This means that the traveling time of a DNA fraction is 38.7 sec., the tray 204 is changed to the collecting vessel 7 36.2 sec after it is detected at the second detection point 403 and the collecting vessel 7 is changed back to the tray 204 41.2 sec. after that time.

Eight kinds of DNA fractions collected by using the method according to this embodiment are amplified at a PCR and forced into capillary-electrophoresis again to verify the accuracy of DNA fraction collection.

The characteristic of this embodiment is in that by allowing the laser beam to irradiate directly onto the columns in the capillary 1 inside of the flow cell 3 for detection, reducing the distance between the second detection point 403 and the capillary end 2 to 2.5 mm, which minimizes an error in timing of DNA fraction collection. Since two-point detection is used, the electrophoretic velocity can be measured between two points in the vicinity of the capillary end 2. Thus, the traveling time can be exactly predicted, enabling DNA fractions to be collected at an accuracy of single nucleotide.

In this embodiment, the electrophoretic velocity is calculated from the difference in time when the sample fractions 5 are detected at the two detection points. Alternatively, the electrophoretic velocities of the molecules may be calculated by directly imaging the behavior of the molecules of the sample fractions 5 at the detection points.

In this embodiment, any kind of proteins or RNAs marked with fluorescent molecules may be used, instead of DNAs, as a sample. In this embodiment, the sample fractions 5 electrophoretically separated may be measured by UV absorption detection, instead of fluorescent detection. For example, an UV laser may be used as a laser source 301 and a biological sample with no fluorescent marking as a sample.

Second Embodiment

A second embodiment is intended to simplify the apparatus configuration by single detection points. Setting of the detection point in the vicinity of the capillary end can minimize an elution time prediction error, allowing correct prediction of traveling times. The system of the second embodiment differs from that of the first embodiment with respect to the detection-part unit 300, and other parts of the system are the same.

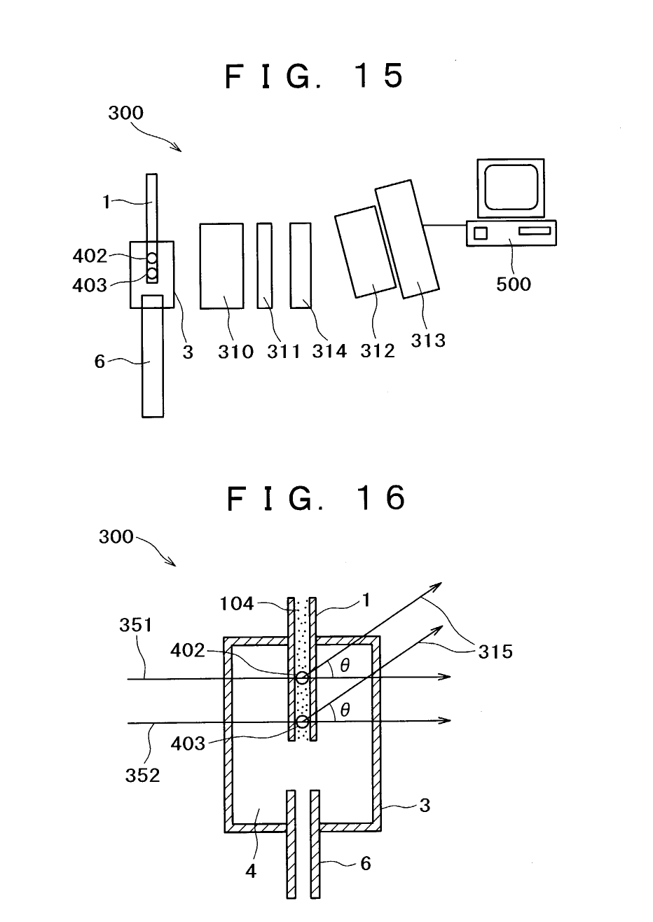

FIG. 13 shows the configuration of the detection-part unit 300 according to the second embodiment. In this embodiment, the laser beam 302 emitted from the laser source 301 is adjusted through the mirror 303 so that it passes through the pinhole 304 and the shutter 307 and irradiated onto the detection point 401 in the capillary 1 inside of the flow cell 3 using the condenser 308.

The configuration inside of the flow cell 3 is the same as that as shown in FIG. 3. Note that the distance between the detection point 401 and the capillary end 2 is 0.12 mm.

FIG. 14 illustrates the procedure for collecting biological fractions. The same procedure is used as that of the first embodiment except for the methods for detection and calculating timing for collection. Only the different processes are described below. Assuming that the time the sample fractions 5 are detected is Tₐ₁₁ and the distance between the sample-injected end 111 of the capillary 1 and the detection point 401 is L, an average Vₐ₁₁ of electrophoretic velocities at an interval of L can be obtained from L/Tₐ₁₁. Assuming that the distance between the detection point 401 and the capillary end 2 is x and the electrophoretic velocity at an interval of x is equal to the Vₐ₁₁, the elution time Ta can be obtained from x/Vₐ₁₁. When x=0.12 (mm), L=360 (mm), and Tₐ₁₁=3000 (sec.), Vₐ₁₁ is 0.12 (mm)/sec. (360/3000), yielding Tₐ of 1.0 (sec.) (0.12/0.12). The sheathe jet flow time T_{b}, during which the sheath flow 16 travels, is equal to that of the first embodiment, 15.7 sec. Accordingly, 16.7 sec. is estimated for the traveling time Tₐ+T_{b} (15.7+1.0) These analyses are performed at the analysis part (the analytical computer 500).

The tray 204 is changed to the collecting vessel 7 in time with the traveling time by issuing the instruction from the analytical computer 500 to collect the target sample fractions.

The characteristic of this aspect of the present invention is in that the distance between the detection point and the capillary end can be made sufficiently shorter by on-column detection inside of the flow cell 3, which reduces the elution time prediction error, resulting in high accuracy of collection.

Third Embodiment

In this embodiment, the illuminant fluorescent light are wavelength-dispersed by a grating so that the biological sample fractions marked with a plural of different fluorescent molecules may be individually identified for detection. The system of the third embodiment differs from that of the first embodiment with respect to the detection-part unit 300, and other parts of the system are the same.

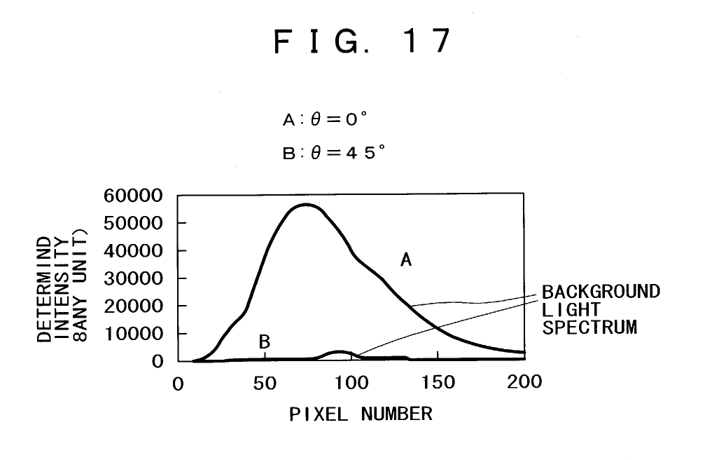

FIG. 15 shows the configuration of the detection-part unit of the third embodiment. The images of the fluorescent light beams the first and second detection points 402 and 403 are formed on the CCD detector 313 using the imaging camera 312 after they pass through the objective camera lens 310, the notch filter 311, and the transmit grating 314. The illuminant fluorescent signals with dispersed wavelengths from each of two detection points are transferred to the analytical computer 500 for data analysis.

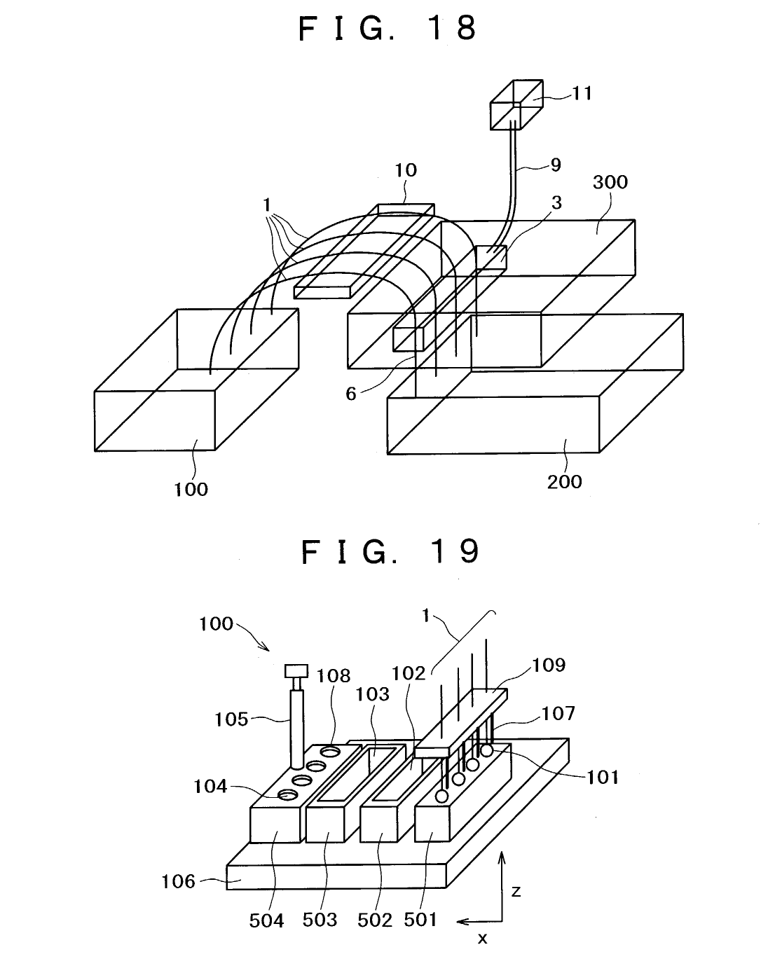

FIG. 16 is a view of the detection-part unit 300 shown in FIG. 15 seen from its left side. As known from FIG. 16, the wavelength-dispersion direction 315, in which the illuminant fluorescent light from the first and second detection points 402 and 403 are wavelength-dispersed, and the wavelength-dispersion angle □ can be changed by inserting the transmittable grating 314. In this case, the wavelength-dispersion angle θ is the angle defined between of the proceeding directions of the laser beams 351 and 352 and the wavelength-dispersion directions, 0°<θ≦90° in this embodiment.

A fluorescent spectrum, of which individual illuminant lights are wavelength-dispersed, can be easily obtained by aligning the pixel-arrangement direction of the CCD detector 313 with the wavelength-dispersion direction. In this embodiment, no beam of 488 nm, the wavelength of the laser beam, comes into the CCD detector 313 because the notch filter 311 shields it. However, Raman scattering of water contained in the sheath liquid 4 and the polymer solution 104 may develop in the vicinity of 584 nm by the 488 nm of laser beam. These Raman-scattering lights come into the CDD detector 313 and superimposed on the fluorescent spectrum, serving as the background spectrum when the fluorescent light is detected. Assuming that the wavelength-dispersion angle θ is 0°, not only the Raman-scattering light beam derived from the polymer solution 104 in the capillary 1 is superimposed on the fluorescent spectrum, but also the Raman-scattering light derived from the sheath liquid 4 on the laser beams 351 and 352 inside of the flow cell 3. The background spectrum developing in this case is shown in FIG. 17. The intensity of the background light becomes much higher, disturbing fluorescent light beam detection. This may leads to deterioration in detection sensitivity and reduction in dynamic range.

In this embodiment, to overcome this problem, θ is set to 45° to prevent the Raman-scattering light derived from the sheath liquid 4 on the laser beams 351 and 352 inside of the flow cell 3 from superimposing on the fluorescent spectrum. The background spectrum, which may develop in this case, is shown in FIG. 17. Thus, since only the Raman-scattering light derived from the polymer solution 104 inside of the capillary 1 at the fluorescent detection points 402 and 403 is superimposed on the fluorescent spectrum, the intensity of the background light can be reduced to one tenth of that shown in FIG. 17. Accordingly, a wider dynamic range of fluorescent lights and detection at a higher sensitivity may be possible.

Alternatively, setting of θ to about 90° to align the wavelength-dispersion direction 315 with the axial direction of the capillary 1 yields the same effect. Note that in this case, fluorescent spectrums from the fluorescent detection points 402 and 403 may be superimposed on the corresponding pixels of the CCD detector 313. To avoid this possible problem, the relative distance between the two detection points is preferably kept sufficiently longer.

Alternatively, one-point detection as shown in FIG. 13 may be used, instead of two-point detection used in this embodiment, to achieve the same effect using the same detection unit configuration.

Fourth Embodiment

This embodiment is intended to increase the throughputs of the capillary electrophoresis and collection processes and a plural of capillaries are configured into an array and electrophoresis and collection can be performed for all individual capillaries concurrently. An example in which the laser beam is guided into the side of the capillary array and the laser is irradiated onto all the capillaries at the same time for fluorescent detection is disclosed in Japanese Patent Application No. 288088/1997.

FIG. 18 shows the configuration of the entire apparatus of this invention. The operation method and the procedure are the same as those of the first embodiment, except that they are performed for each capillary.

The components are the same as those of the first embodiment. Four capillaries 1 are arranged into an capillary array. The four capillaries 1 are arranged so that they are not overlapped to uniformly adjust the temperature of each capillary. The polyimide-coated quartz tubes (Polymicro) with an outer diameter of 200 μm, an inner diameter of 100 μm, and a length of 36 cm are used.

FIG. 19 shows the details of the sample-injected side unit 100. The same number of samples 101 are prepared as that of capillaries, the buffer 102 is poured into the buffer vessel 502, pure water 103 is into the pure water vessel 503, the polymer solution 104 is into the polymer vessel 504, and the syringe 105 is set. These vessels are disposed on the base 106, which is movable in the X and Y directions.

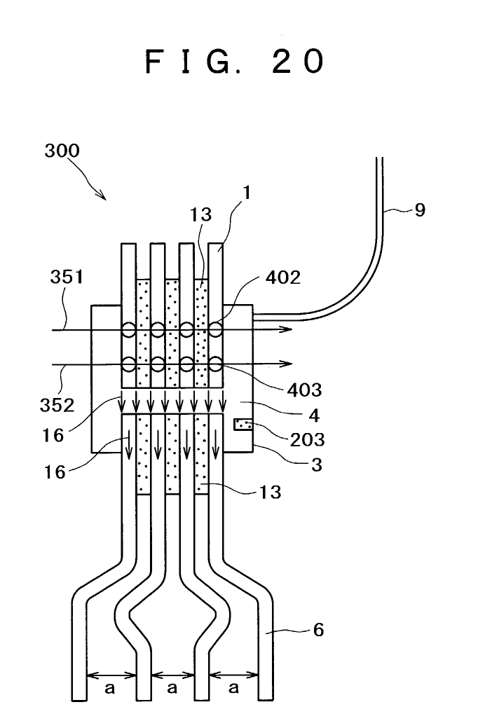

Now, with reference to FIG. 20, the configuration of the inside of the flow cell 3 is shown. Each of capillaries 1 is arranged vertically against the laser beam on the same plane as shown in the figure. To condense the first and second laser beams 351 and 352 at the first and second detection points 402 and 403 of each capillary 1, rod lenses 13 are inserted between the capillaries. Quartz lenses with a refraction index of 1.46, which is the same as that of the capillary 1, are used as the rod lenses 13. The sheath liquid with a refraction index of 1.33 and the polymer solution 104 with a refraction index of 1.41 are used. Under these conditions, the laser beam is effectively condensed into all of four capillaries (Kambara et al., Electrophoresis 1999 20 539-546). The collecting tube 6 is disposed at the opposite position of each capillary 1 inside of the flow cell. The polyimide-coated quartz tube with a length of 70 mm is used as a collecting tube 6. The rod lens 13 is inserted between the collecting tubes 6. The end-side electrode 203 is submerged in the sheath liquid 4, which serves as the ground, inside of the flow cell 3. The configuration of the remaining detection-part unit is the same as that of the third embodiment. Note that θ is set to 90° to set the wavelength-dispersion direction 315 almost vertically against the laser beam. In this case, to avoid superimposition of signals from the two detection points, after wavelength-dispersed, on the CCD detector 313, the relative distance between the detection points is kept longer. The collecting tubes 6 are arranged on a straight line at an interval a of 8 mm at the collecting point 8 and bent so that the total lengths of the collecting tubes 6.

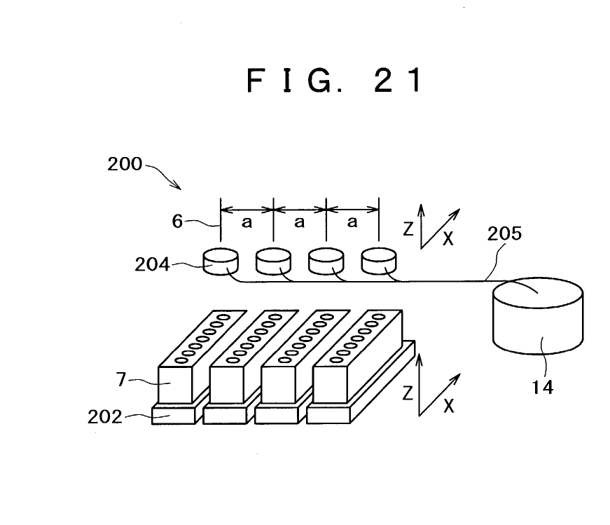

FIG. 21 shows the configuration of the end-side unit. The collecting vessels 7 are arranged correspondingly to the associated collecting tubes 6 at an interval a of 8 mm. To enable biological fractions to be individually collected from each capillary, the collecting vessel 7 is disposed on each of four collecting vessel bases 202, resulting in the capillaries being individually movable in the X and Y directions. The target sample fractions 5 are collected into the collecting vessels 7 by moving the tray 204 based on the data from analysis by the analytical computer. Since the sample fractions 5 are detected and the driving instruction is issued for each capillary, a plural of collection processes of the sample fractions can be performed concurrently.

As mentioned above, by integrating a plural of capillaries into one array, the throughput of collection can be improved by the same scale as the number of capillaries.

Fifth Embodiment

In this embodiment, to improve the throughputs of the capillary-electrophoresis and collection processes, a plural of capillaries are integrated into one array so that the electrophoresis and collection processes can be performed for all capillaries concurrently. An example is given in which laser irradiation and fluorescent detection are sequentially performed for all capillaries by scanning the laser beam in Anal. Chem. 1992 Vol. 64, 2149-2154.

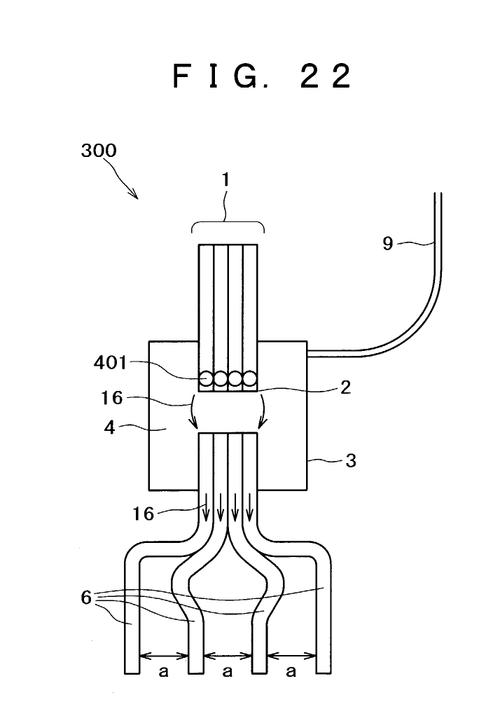

The fundamental configuration of the apparatus is based on the fourth embodiment. The procedure is the same as that of the second embodiment. Note that the detection-part unit 300 is different from that of the embodiments. First, the configuration of the inside of the flow cell 3 is shown in FIG. 22. Four capillaries 1 are arranged on the same plane closely each other. One detection point is set 0.12 mm apart from the capillary end 2 for each capillary. Like those of the second embodiment, the detection point 401 is set closely to the capillary end 2 to minimize the elution time prediction error. The laser beam 302 is irradiated on the capillaries from the direction almost vertical against the paper surface of FIG. 22 and fluorescent detection is performed from the same side as that of beam irradiation.

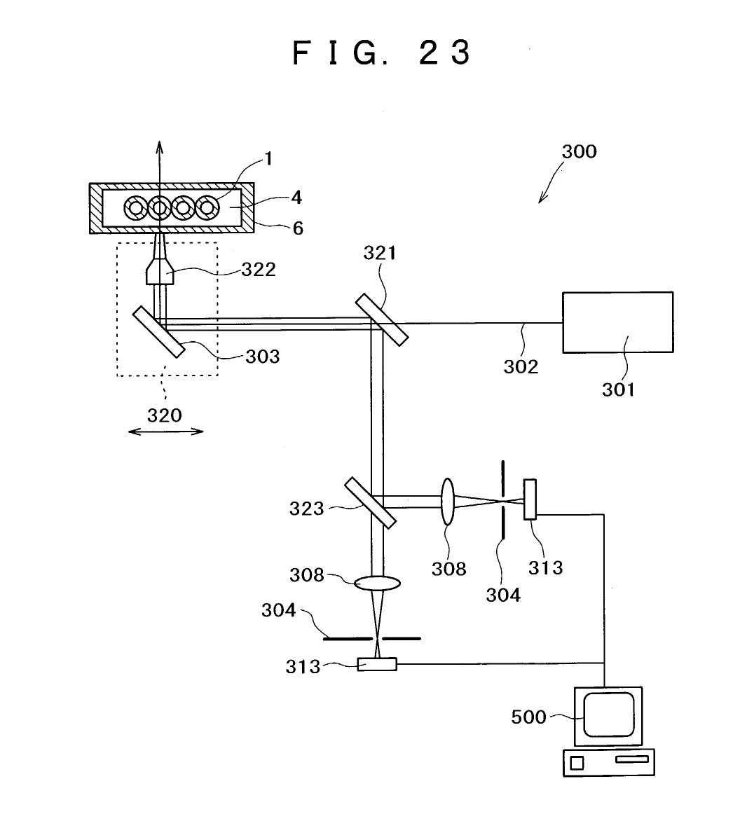

FIG. 23 shows a schematic view of the configuration of the detection-part unit 300. The laser beam 302 emitted from the laser source 301 passing through a first dichroic mirror 321 is reflected on the mirror 303, condensed by the objective lens 322, and irradiated onto the detection point of the capillary 1. The first dichroic mirror 321 transmits the laser beam with a wavelength of 488 nm while reflecting the laser beam with a wavelength of 520 nm or longer. The mirror 303 and the objective lens 322 make up the driving unit 320, which reciprocate at a high speed in the same direction as that of the capillary array. This enables the laser beam 302 to scan-irradiate onto individual capillaries sequentially. The illuminant fluorescent light emitted from the detection point 401 of the capillary 1 are condensed at the same objective lens 322. The fluorescent light is reflected on the mirror 303 and then on the first dichroic mirror 321. Next, the light beam, which is split into two fluorescent wavelengths on the second dichroic mirror, passes through the condensing lens 308 and then the pinhole 304, and finally received at the CCD detector 313. In this configuration, two kinds of biological fractions with different fluorescent wavelengths can be detected. The detected signals are transferred to the analytical computer 500 for data analysis. The configuration of remaining components is the same as that of the fourth embodiment and the procedure for collecting biological fractions is the same as that of the second embodiment.

A mentioned above, integration of capillaries into one array improves the throughput of detection efficiency by the scale corresponding to the number of capillaries.

In the foregoing specification, the invention has been described with reference to specific embodiments thereof. It will, however, be evident that various modifications and changes may be made thereto without departing from the broader spirit and scope of the invention. The specification and drawings are, accordingly, to be regarded in an illustrative rather than a restrictive sense.

What is claimed is:

1. A system for fraction collection for capillary electrophoresis, the system comprising:
a capillary configured to electrophoretically separate biological fractions into separated biological fractions, and further configured to discharge the separated biological fractions from a capillary end;
a flow cell surrounding the capillary end, wherein the flow cell is configured to move the separated biological fractions discharged from the capillary end into a collecting vessel;
an irradiator configured to irradiate a light beam onto the capillary and the flow cell, wherein the light beam causes emitted light from inside of the capillary; and
a detector configured to detect the emitted light.

2. The system of claim 1, wherein the separated biological fractions are at least one of:
DNA;
RNA; and
protein.

3. The system of claim 1, wherein a distance between an intersection of the capillary with the light beam and the capillary end is 2.6 mm or less.

4. The system of claim 1, wherein the light beam is irradiated onto the capillary and onto the flow cell at a detection point, wherein the system further comprises:
an analyzer device including
a velocity detector device configured to calculate an average electrophoretic velocity of the separated biological fractions at the detection point,
a timer device configured to calculate a time Ta required for the separated biological fractions to travel from the detection point to the capillary end based on the average electrophoretic velocity, and further configured to calculate a time Tb required for the separated biological fractions to travel from the capillary end to the collection vessel based on a flow rate of a sheath fluid flowing into the flow cell, and
a predictor device configured to predict a required traveling time for the separated biological fractions to travel from the detection point to the collection vessel based on a sum of the time Ta and the time Tb; and
a driver device configured to drive the collecting vessel according to the required traveling time.

5. The system of claim 1, wherein the irradiator is further configured to irradiate the light beam onto the capillary and onto the flow cell at two detection points.

6. The system of claim 5, wherein a distance between the capillary end and a detection point nearest the capillary end is 2.6 mm or less.

7. The system of claim 5, further comprising:
an analyzer device including
a velocity dector device configured to calculate an average electrophoretic velocity of the separated biological fractions between the two detection points,
a timer device configured to calculate a time Ta required for the separated biological fractions to travel from a detection point nearest the capillary end to the capillary end based on the average electrophoretic velocity, and further configured to calculate a time Tb required for the separated biological fractions to travel from the capillary end to the collection vessel based on a flow rate of a sheath fluid flowing into the flow cell, and
a predictor device configured to predict a required traveling time for the separated biological fractions to travel from the detection point nearest the capillary end to the collection vessel based on a sum of the time Ta and the time Tb; and
a driver device configured to drive the collecting vessel according to the required traveling time.

8. The system of claim 1, wherein the separated biological fractions are labeled with a fluorescent dye, wherein the emitted light is emitted from the fluorescent dye, wherein the detector is further configured to detect the emitted light.

9. The system of claim 1, wherein the irradiator is further configured to irradiate an ultraviolet light beam so that an absorption of the ultraviolet light beam by the separated biological fractions may be detected.

10. The system of claim 1, further comprising a plurality of other capillaries each configured like the capillary, wherein the system is further configured to be applied to the plurality of other capillaries.

11. The system of claim 1, further comprising a plurality of other capillaries arrayed in the flow cell on a substantially same plane as the capillary, wherein the light beam is configured to be introduced from a side of the substantially same plane and to irradiate all capillaries.

12. The system of claim 1, further comprising:
a plurality of other capillaries arrayed in the flow cell on a substantially same plane as the capillary; and
a plurality of light-condensing devices on the same plane, wherein the light-condensing devices arrayed with the plurality of other capillaries in the substantially same plane, wherein the light-condensing devices are configured so that the light beam is introduced from a side of the substantially same plane to irradiate all capillaries.

13. The system of claim 12, wherein each of the plurality of light-condensing devices is a rod lens made of glass.

14. The system of claim 1, further comprising:
a plurality of other capillaries arrayed in the flow cell on a substantially same plane as the capillary; and
a scanner device configured to scan the light beam and the detector in the direction in which the capillaries are arrayed.

15. The system of claim 1, wherein the irradiator is further configured to irradiate the light beam in an almost vertical direction, wherein the detector is further configured to detect the light beam in the almost vertical direction, wherein the system further comprises a dispersing device configured to disperse a wavelength of the light beam into a wavelength-dispersed light beam, wherein the detector is further configured to detect the wavelength-dispersed light beam, wherein an angle between a direction of the light beam and a direction of the wavelength-dispersed light beam is between 0 degrees and 90 degrees.

16. The system of claim 1, wherein the irradiator is further configured to irradiate the light beam in an almost vertical direction, wherein the detector is further configured to detect the light beam in the almost vertical direction, wherein the system further comprises a dispersing device configured to disperse a wavelength of the light beam into a wavelength-dispersed light beam, wherein the detector is further configured to detect the wavelength-dispersed light beam, wherein and an angle between a direction of the light beam and a direction of the wavelength-dispersed light beam is almost 90 degrees.

17. A method for fraction collection for capillary electrophoresis, the method comprising:
separating biological fractions in a capillary into separated biological fractions;
moving the separated biological fractions discharged from the capillary end by injecting a sheath fluid into the flow cell surrounding the capillary end;
irradiating a light beam at the capillary and the flow cell, wherein the light beam cause emitted light from the capillary;
detecting the emitted light; and
collecting into individual collecting vessels the separated biological fractions discharged from the flow cell.

18. The method of claim 17, wherein before the step of separating the biological fractions, the method further comprises introducing an electrolyte solution into capillary.
